(19) United States
(12) Patent Application Publication
(10) Pub. No.: US 2021/0343373 A1
(43) Pub. Date: Nov. 4, 2021

(54) SEQUENCE ALIGNMENT FILTERING PROCESSING METHOD, SYSTEM AND DEVICE, AND READABLE STORAGE MEDIUM

(71) Applicant: ZHENGZHOU YUNHAI INFORMATION TECHNOLOGY CO., LTD., Zhengzhou, Henan (CN)

(72) Inventors: Jian ZHAO, Zhengzhou, Henan (CN); Hongzhi SHI, Zhengzhou, Henan (CN); Xingchen CUI, Zhengzhou, Henan (CN); Yunfeng YIN, Zhengzhou, Henan (CN)

(73) Assignee: ZHENGZHOU YUNHAI INFORMATION TECHNOLOGY CO., LTD., Zhengzhou, Henan (CN)

(21) Appl. No.: 17/280,926

(22) PCT Filed: Aug. 30, 2019
(86) PCT No.: PCT/CN2019/103720

§ 371 Date: Mar. 29, 2021

(30) Foreign Application Priority Data
Jan. 31, 2019 (CN) 201910098868.2

(51) Int. Cl. G16B 30/10

(52) U.S. Cl. CPC G16B 30/10

(57) ABSTRACT

A filter processing method for a sequence alignment is provided. The method includes: searching for absolute locations of all seeds of a to-be-aligned sequence in a reference sequence; performing segmentation on the absolute locations of the seeds to obtain relative locations of the seeds; dividing the reference sequence into multiple reference sub-sequences and establishing a mapping relationship between a relative location of each seed and a corresponding reference sub-sequence; determining a reference sub-sequence to which each seed belongs and counting the occurrence numbers of the seeds in each reference sub-sequence; filtering out a reference sub-sequence that does not meet a preset condition to obtain a target reference sub-sequence; and recovering a real CAL based on a difference between a relative location and an absolute location of each seed in the target reference sub-sequence.

This application claims the priority to Chinese Patent Application No. 201910098868.2, titled “SEQUENCE ALIGNMENT FILTERING PROCESSING METHOD, SYSTEM AND DEVICE, AND READABLE STORAGE MEDIUM”, filed on Jan. 31, 2019 with the China National Intellectual Property Administration (CNIPA), which is incorporated herein by reference in its entirety.

### FIELD

The present disclosure relates to the technical field of computers, and in particular to a filter processing method for a sequence alignment, a system and a device thereof and a computer-readable storage medium.

### BACKGROUND

With rapid development of biological gene detection technology, prevention and treatment of individual disease in advance are increasingly mature, by means of extracting individual genes for gene sequence alignment, predicting possibility of suffering from a variety of diseases, and determining genes of the individual disease. Currently, human gene pool has about 3 billion base pairs. It takes several days to complete the individual gene sequence alignment by using a universal computer software processing platform. The conventional CPU processing platform cannot meet the requirements in acquiring results of the gene sequence alignment rapidly and in a real-time manner. With increase of requirements for computing performance of the platform, various high-performance accelerators, such as GPU and FPGA are gradually applied to the gene sequence alignment.

The algorithm of the gene sequence alignment mainly includes two stages: searching for seeds and expansion. In order to improve accuracy of the gene sequence alignment, it is required to find a location of a seed of a to-be-aligned sequence in a reference sequence. Because of alignment processing for a large number of invalid locations, performance of a system for the gene sequence alignment is greatly degraded.

Therefore, it is required to perform filter processing on the seed found in a previous stage, to filter out as many invalid locations as possible, so as to reduce workloads of subsequent expansion, and ensure the accuracy of the system for the sequence alignment.

### SUMMARY

In view of this, a filter processing method for a sequence alignment, a system and a device thereof, and a computer-readable storage medium are provided according to the present disclosure, to reduce workloads of subsequent expansion and improve working efficiency. The specific solutions are as follows.

A filter processing method for a sequence alignment is provided. The method includes:
searching for absolute locations of all seeds of a to-be-aligned sequence in a reference sequence;
performing segmentation on the absolute locations of all the seeds in the reference sequence, to obtain relative locations of all the seeds;
dividing the reference sequence into multiple reference sub-sequences in advance, and establishing a mapping relationship between a relative location of each seed and a reference sub-sequence corresponding to the seed;
determining a reference sub-sequence to which each seed belongs according to a feature identifier of the seed and the mapping relationship, and counting the numbers of occurrences of the seeds in each reference sub-sequence;
filtering out a reference sub-sequence that does not meet a preset condition based on the numbers of occurrences of the seeds in each reference sub-sequence, to obtain a target reference sub-sequence meeting the preset condition; and
recovering a real CAL based on a difference between a relative location and an absolute location of each seed in the target reference sub-sequence.

In an embodiment, the process of determining a reference sub-sequence to which each seed belongs according to a feature identifier of the seed and the mapping relationship includes:
calculating a hash value of each seed; and
determining the reference sub-sequence to which each seed belongs from a filtered hash table storing the mapping relationship, with the hash value of each seed as an address.

In an embodiment, the process of filtering out a reference sub-sequence that does not meet a preset condition based on the numbers of occurrences of the seeds in each reference sub-sequence, includes:
setting a dynamic filtering threshold according to the numbers of occurrences of the seeds in each reference sub-sequence, a mean value of the numbers of occurrences, and/or a maximum descending gradient of the numbers of occurrences; and
filtering out a reference sub-sequence that does not meet the dynamic filtering threshold.

A filter processing system for a sequence alignment is further provided according to the present disclosure. The filtering system includes: an absolute location searching module, an absolute location segmentation module, a mapping relationship establishing module, an occurrence number counting module, a sub-sequence filtering module, and a CAL recovering module. The absolute location searching module is configured to search for absolute locations of all seeds of a to-be-aligned sequence in a reference sequence. The absolute location segmentation module is configured to perform segmentation on the absolute locations of all the seeds in the reference sequence, to obtain relative locations of all the seeds. The mapping relationship establishing module is configured to divide the reference sequence into multiple reference sub-sequences in advance, and establish a mapping relationship between a relative location of each seed and a reference sub-sequence corresponding to the seed. The occurrence number counting module is configured to determine a reference sub-sequence to which each seed belongs according to a feature identifier of the seed and the mapping relationship, and count the numbers of occurrences of the seeds in each reference sub-sequence. The sub-sequence filtering module is configured to filter out a reference sub-sequence that does not meet a preset condition based on the numbers of occurrences of the seeds in each reference sub-sequence, to obtain a target reference sub-sequence meeting the preset condition. The CAL recovering module is configured to recover a real CAL based on a difference between a relative location and an absolute location of each seed in the target reference sub-sequence.

In an embodiment, the occurrence number counting module includes: a hash value calculating unit and a determining unit. The hash value calculating unit is configured to calculate a hash value of each seed. The determining unit is configured to determine the reference sub-sequence to which each seed belongs from a filtered hash table storing the mapping relationship, with the hash value of each seed as an address.

In an embodiment, the sub-sequence filtering module includes: a threshold setting unit and a filtering unit. The threshold setting unit is configured to set a dynamic filtering threshold according to the numbers of occurrences of the seeds in each reference sub-sequence, a mean value of the numbers of occurrences, and/or a maximum descending gradient of the numbers of occurrences. The filtering unit is configured to filter out a reference sub-sequence that does not meet the dynamic filtering threshold.

A filter processing device for a sequence alignment is further provided according to the present disclosure. The filtering processing device includes a memory and a processor. The memory is configured to store a computer program. The processor is configured to execute the computer program to perform the filter processing method for a sequence alignment described above.

A computer-readable storage medium storing a computer program is further provided according to the present disclosure. The computer program is executed by a processor to perform the filter processing method for a sequence alignment described above.

The filter processing method for a sequence alignment according to the present disclosure includes: searching for absolute locations of all seeds of a to-be-aligned sequence in a reference sequence; performing segmentation on the absolute locations of all the seeds in the reference sequence, to obtain relative locations of all the seeds; dividing the reference sequence into multiple reference sub-sequences in advance, and establishing a mapping relationship between a relative location of each seed and a reference sub-sequence corresponding to the seed; determining a reference sub-sequence to which each seed belongs according to a feature identifier of the seed and the mapping relationship, and counting the numbers of occurrences of the seeds in each reference sub-sequence; filtering out a reference sub-sequence that does not meet a preset condition based on the numbers of occurrences of the seeds in each reference sub-sequence, to obtain a target reference sub-sequence meeting the preset condition; and recovering a real CAL based on a difference between a relative location and an absolute location of each seed in the target reference sub-sequence.

According to the present disclosure, segmentation is performed on the absolute locations of the seeds of the to-be-aligned sequence in the reference sequence, the numbers of occurrences of all the seeds of the to-be-aligned sequence in each reference sub-sequence are counted, and the dynamic filtering threshold is set based on the counted numbers of occurrences of the seeds in each reference sub-sequence, so as to filter out as many invalid locations as possible, thereby reducing the workloads of subsequent expansion, ensuring the alignment accuracy of the system for the sequence alignment, and improving the working efficiency.

### BRIEF DESCRIPTION OF THE DRAWINGS

In order to illustrate technical solutions in embodiments of the present disclosure or in the conventional technology more clearly, drawings used in the description of the embodiments or the conventional technology are briefly introduced hereinafter. It is apparent that the drawings in the following description show only embodiments of the present disclosure, and other drawings may be obtained by those ordinary skilled in the art based on the provided drawings without creative efforts.

FIG. 1 is a schematic flowchart of a filter processing method for a sequence alignment according to an embodiment of the present disclosure; and

FIG. 2 is a schematic structural diagram of a filter processing system for a sequence alignment according to an embodiment of the present disclosure.

### DETAILED DESCRIPTION

Hereinafter, technical solutions in the embodiments of the present disclosure will be described clearly and completely in conjunction with the drawings in the embodiments of the present disclosure. It is apparent that the embodiments in the following description are only some embodiments of the present disclosure, rather than all of the embodiments. Based on the embodiments of the present disclosure, all other embodiments obtained by those skilled in the art without any creative work fall within the scope of protection of the present disclosure.

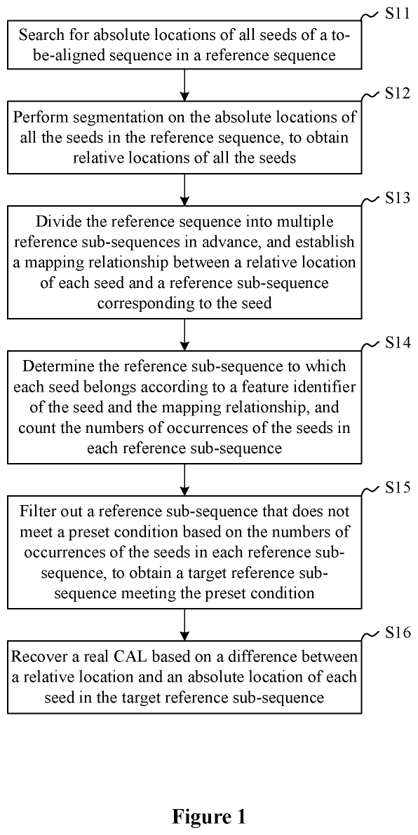

A filter processing method for a sequence alignment is provided according to an embodiment of the present disclosure. As shown in FIG. 1, the method includes the following steps S11 to S16.

In step S11, absolute locations of all seeds of a to-be-aligned sequence are searched for in a reference sequence.

Locations of all the seeds of the to-be-aligned sequence in the reference sequence are searched for. The location is defined as the absolute location, to facilitate subsequent recovery of a candidate alignment location (CAL) in a reference sub-sequence.

In step S12, segmentation is performed on the absolute locations of all the seeds in the reference sequence, to obtain relative locations of all the seeds.

Segmentation is performed on the absolute locations of all the seeds in the reference sequence, to extract the absolute locations of all the seeds from the reference sequence, so as to obtain the relative locations of all the seeds outside the reference sequence. The absolute locations of the seeds of the to-be-aligned sequence in the reference sequence can be quickly found by extracting the absolute locations of the seeds from the reference sequence.

A size of a segment depends on a length of the to-be-aligned sequence and an encoding format of the to-be-aligned sequence. For example, the size of the segment may be set to be 256 bits, that is, a size of each of the reference sub-sequences is 256 bits and a size of the recovered CAL is an integer multiple of 256.

In step S13, the reference sequence is divided into multiple reference sub-sequences in advance, and a mapping relationship between a relative location of each seed and a reference sub-sequence corresponding to the seed is established.

The number of the reference sub-sequences divided from the reference sequence is set in advance. The number of the reference sub-sequences is greater, overlapping probability of the absolute locations of the reference sub-sequences is less, and a potential alignment loss is less. Certainly, the number of the reference sub-sequences is excessive, resulting in increase of operation time. Therefore, the specific number of the reference sub-sequences may be reasonably set according to alignment accuracy and performance in actual applications.

The mapping relationship between the relative location of each seed and the reference sub-sequence corresponding to the seed is established, so that the reference sub-sequence corresponding to the seed is found according to a feature identifier of the seed. The mapping relationship may be stored in a form of a table, or may also be stored in a form of a file or data. A storage form of the mapping relationship is not limited herein.

In step S14, a reference sub-sequence to which each seed belongs is determined according to the feature identifier of the seed and the mapping relationship, and the number of occurrences of the seed in each reference sub-sequence is counted.

A unique identifier may be added to each seed to serve as a feature identifier for indicating an identity of the seed. The feature identifier may be a code having a one-to-one correspondence with the seed, or may be a hash value obtained by performing hash calculation for the seed.

It should be noted that the absolute location of each seed in the reference sequence has a direct correspondence with the relative location of the seed. Therefore, the relative location of the seed may be found according to the feature identifier of the seed. Further, the reference sub-sequence to which each seed belongs is determined according to the feature identifier of the seed and the mapping relationship.

Each of the reference sub-sequences may include multiple seeds. The reference sub-sequence including more seeds is more similar to the to-be-aligned sequence, and the accuracy of the subsequent alignment is higher. Therefore, the number of the occurrences of the seed in each reference sub-sequence is counted to facilitate subsequent filtering.

In step S15, a reference sub-sequence that does not meet a preset condition is filtered out based on the number of occurrences of the seed in each reference sub-sequence, to obtain a target reference sub-sequence meeting the preset condition.

In order to reduce alignment of invalid locations and workloads of subsequent expansion, most of the reference sub-sequences that obviously do not meet requirements may be filtered out in advance, to reduce the invalid locations and the workloads of subsequent expansion, thereby improving working efficiency. The preset condition is set based on the number of occurrences of the seed in each reference sub-sequence. The reference sub-sequences are filtered according to the preset condition, and only the target reference sub-sequence meeting the preset condition is reserved for subsequent CAL recovery.

It should be understood that, the preset condition may be set based on the number of the occurrences of the seed in each reference sub-sequence. For example, the preset condition may be a threshold of a mean value obtained from the numbers of occurrences or may be other values calculated from the numbers of occurrences of the seeds in each reference sub-sequence. The preset condition is set according to actual application scenes.

In step S16, a real CAL is recovered based on a difference between a relative location and an absolute location of each seed in the target reference sub-sequence.

After the relative location and the absolute location of each seed are obtained, the difference between the relative location and the absolute location of the seed is recorded in advance, to recover the real CAL. For example, assuming the size of the segment is 256 and the absolute location of the seed is 258. Since the size of the CAL is an integral multiple of the size of the segment, the relative location of the seed is 2 and the difference is 256. The real CAL is recovered based on 2+256.

It can be seen that in the embodiment of the present disclosure, segmentation is performed on the absolute locations of the seeds of the to-be-aligned sequence in the reference sequence, the number of occurrences of the seeds of the to-be-aligned sequence in each reference sub-sequence is counted, and the dynamic filtering threshold is set according to the counted numbers of occurrences of the seeds in each reference sub-sequence, so as to filter out as many invalid locations as possible, thereby reducing the workloads of subsequent expansion, ensuring the alignment accuracy of the system for the sequence alignment, and improving the working efficiency.

Another filter processing method for a sequence alignment is provided according to an embodiment of the present disclosure. Compared with the above embodiment, the technical solutions are further illustrated and optimized according to the embodiment.

The process of determining a reference sub-sequence to which each seed belongs according to the feature identifier of the seed and the mapping relationship in step S14 may include step S141 and step S142.

In step S141, a hash value of each seed is calculated.

In step S142, the reference sub-sequence to which each seed belongs is determined from a filtered hash table storing the mapping relationship, with the hash value of each seed as an address.

The feature identifier of the seed may be the hash value. The mapping relationship may be stored in a form of the filtered hash table. The hash value of the seed serves as the address to directly search the filtered hash table, so that the reference sub-sequence to which each seed belongs is determined according to the mapping relationship in the filtered hash table.

The process of filtering out a reference sub-sequence that does not meet a preset condition based on the number of occurrences of the seed in each reference sub-sequence in the above step S15 may include steps S151 and S152.

The number of the occurrence of the seed in the reference sub-sequence is counted by performing segmentation on the absolute location of the seed in the reference sub-sequence and then searching the hash table. Since the location of the occurrence of the seed in the reference sub-sequence is uncertain and the occurrence number may be large, the hash table is designed to allow collision.

In step S151, the dynamic filtering threshold is set according to the numbers of occurrences of the seeds in each reference sub-sequence, a mean value of the numbers of occurrences, and/or a maximum descending gradient of the numbers of occurrences.

The filtering threshold is set preferably based on the descending gradient of the counted numbers of occurrences of the seeds in the reference sub-sequence. In a case that the descending gradient reaches a predetermined value, CAL less than a counted number of occurrences of the seeds in a current reference sub-sequence is directly filtered out. In a case that the descending gradient does not reach the predetermined value, CAL less than a mean value of the counted numbers of occurrences of the seeds in a current reference sub-sequence is directly filtered out. In a case that a maximum value of the numbers of occurrences of the seeds in the reference sub-sequence is significantly greater than the mean value of the counted numbers of occurrences of the seeds in the reference sub-sequence, CAL less than the maximum value of the counted numbers of occurrences of the seeds in the reference sub-sequence is directly filtered out. Alternatively, the filtering threshold is set based on the numbers of occurrences of the seeds in each reference sub-sequence, the mean value of the numbers of occurrences and the maximum descending gradient of the numbers of occurrences, or other factors according to the requirements of actual application.

The descending gradient of the counted number of occurrences of the seeds in the reference sub-sequence is a difference between two adjacent counted numbers of occurrences after the counted numbers of occurrences of the seeds in all the reference sub-sequences are ranked in a descending order.

In step S152, a reference sub-sequence that does not meet the dynamic filtering threshold is filtered out.

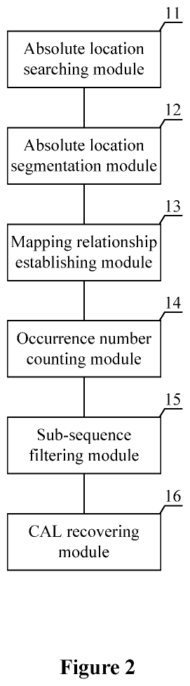

A filter processing system for a sequence alignment is further provided according to an embodiment of the present disclosure. As shown in FIG. 2, the system includes an absolute location searching module 11, an absolute location segmentation module 12, a mapping relationship establishing module 13, an occurrence number counting module 14, a sub-sequence filtering module 15 and a CAL recovering module 16.

The absolute location searching module 11 is configured to search for absolute locations of all seeds of a to-be-aligned sequence in a reference sequence.

The absolute location segmentation module 12 is configured to perform segmentation on the absolute locations of all the seeds in the reference sequence, to obtain relative locations of all the seeds.

The mapping relationship establishing module 13 is configured to divide the reference sequence into multiple reference sub-sequences in advance, and establish a mapping relationship between a relative location of each seed and a reference sub-sequence corresponding to the seed.

The occurrence number counting module 14 is configured to determine the reference sub-sequence to which each seed belongs according to a feature identifier of the seed and the mapping relationship, and count the number of occurrences of the seed in each reference sub-sequence.

The sub-sequence filtering module 15 is configured to filter out a reference sub-sequence that does not meet a preset condition based on the numbers of occurrences of the seeds in each reference sub-sequence, to obtain a target reference sub-sequence meeting the preset condition.

The CAL recovering module 16 is configured to recover a real CAL based on a difference between a relative location and an absolute location of each seed in the target reference sub-sequence.

The occurrence number counting module 14 may include a hash value calculating unit and a determining unit.

The hash value calculating unit is configured to calculate a hash value of each seed.

The determining unit is configured to determine the reference sub-sequence to which each seed belongs from a filtered hash table storing the mapping relationship, with the hash value of each seed as an address.

The sub-sequence filtering module 15 may include a threshold setting unit and a filtering unit.

The threshold setting unit is configured to set a dynamic filtering threshold according to the numbers of occurrences of the seeds in each reference sub-sequence, a mean value of the numbers of occurrences, and/or a maximum descending gradient of the numbers of occurrences.

The filtering unit is configured to filter out a reference sub-sequence that does not meet the dynamic filtering threshold.

In addition, a filter processing device for a sequence alignment is further provided according to an embodiment of the present disclosure. The device includes a memory and a processor.

The memory is configured to store a computer program.

The processor is configured to execute the computer program to perform the filter processing method for a sequence alignment described above.

In addition, a computer-readable storage medium storing a computer program is further provided according to an embodiment of the present disclosure. The computer program is executed by a processor to perform the filter processing method for a sequence alignment described above.

Finally, it should be further noted that, the relationship terminologies such as “first”, “second” and the like in the present disclosure, are only used herein to distinguish one entity or operation from another, rather than to necessitate or imply that the actual relationship or order exists between the entities or operations. Furthermore, terms of “include”, “comprise” or any other variants are intended to be non-exclusive. Therefore, a process, a method, an article or a device including a plurality of elements includes not only the elements but also other elements that are not enumerated, or also include the elements inherent for the process, the method, the article or the device. Unless expressively limited otherwise, the statement “comprising (including) one . . . ” does not exclude the case that other similar elements may exist in the process, the method, the article or the device.

It may be known by those skilled in the art that, units and steps in the device and method described in conjunction with the embodiments disclosed herein can be implemented by electronic hardware, computer software or a combination thereof. In order to clearly illustrate interchangeability of the hardware and the software, steps and composition of each embodiment have been described generally in view of functions in the above specification. Whether the function is executed in a hardware way or in a software way depends on application of the technical solution and design constraint condition. Those skilled in the art can implement the described function by using different method for each application, and such implementation should not be considered to go beyond the scope of the present disclosure.

The filter processing method for a sequence alignment, the system and the device thereof and the computer-readable storage medium according to the present disclosure are described in detail above. Specific examples are used herein to explain the principle and the implementation of the present disclosure. The above description of the embodiments is only used to help understanding the methods and core concept of the present disclosure. In addition, for those skilled in the art, the specific implementation and application scope may be changed according to the concept of the present disclosure. In summary, the content of the present disclosure is not intended to limit the present disclosure.

1. A filter processing method for a sequence alignment, comprising:
searching for absolute locations of all seeds of a to-be-aligned sequence in a reference sequence;
performing segmentation on the absolute locations of all the seeds in the reference sequence, to obtain relative locations of all the seeds;
dividing the reference sequence into a plurality of reference sub-sequences in advance, and establishing a mapping relationship between a relative location of each seed and a reference sub-sequence corresponding to the seed;
determining a reference sub-sequence to which each seed belongs according to a feature identifier of the seed and the mapping relationship, and counting the numbers of occurrences of the seeds in each reference sub-sequence;
filtering out a reference sub-sequence that does not meet a preset condition based on the numbers of occurrences of the seeds in each reference sub-sequence, to obtain a target reference sub-sequence meeting the preset condition; and
recovering a real CAL based on a difference between a relative location and an absolute location of each seed in the target reference sub-sequence.

2. The filter processing method for a sequence alignment according to claim 1, wherein the determining a reference sub-sequence to which each seed belongs according to a feature identifier of the seed and the mapping relationship comprises:
calculating a hash value of each seed; and
determining the reference sub-sequence to which each seed belongs from a filter hash table storing the mapping relationship, with the hash value of each seed as an address.

3. The filter processing method for a sequence alignment according to claim 1, wherein the filtering out a reference sub-sequence that does not meet a preset condition based on the numbers of occurrences of the seeds in each reference sub-sequence comprises:
setting a dynamic filtering threshold according to the numbers of occurrences of the seeds in each reference sub-sequence, a mean value of the numbers of occurrences, and/or a maximum descending gradient of the numbers of occurrences; and
filtering out a reference sub-sequence that does not meet the dynamic filtering threshold.

4. A filter processing system for a sequence alignment, comprising:
an absolute location searching module configured to search for absolute locations of all seeds of a to-be-aligned sequence in a reference sequence;
an absolute location segmentation module configured to perform segmentation on the absolute locations of all the seeds in the reference sequence, to obtain relative locations of all the seeds;
a mapping relationship establishing module configured to divide the reference sequence into a plurality of reference sub-sequences in advance, and establish a mapping relationship between a relative location of each seed and a reference sub-sequence corresponding to the seed;
an occurrence number counting module configured to determine a reference sub-sequence to which each seed belongs according to a feature identifier of the seed and the mapping relationship, and count the numbers of occurrences of the seeds in each reference sub-sequence;
a sub-sequence filtering module configured to filter out a reference sub-sequence that does not meet a preset condition based on the numbers of occurrences of the seeds in each reference sub-sequence, to obtain a target reference sub-sequence meeting the preset condition; and
a CAL recovering module configured to recover a real CAL based on a difference between a relative location and an absolute location of each seed in the target reference sub-sequence.

5. The filter processing system for a sequence alignment according to claim 4, wherein the occurrence number counting module comprises:
a hash value calculating unit configured to calculate a hash value of each seed; and
a determining unit configured to determine the reference sub-sequence to which each seed belongs from a filtered hash table storing the mapping relationship, with the hash value of each seed as an address.

6. The filter processing system for a sequence alignment according to claim 4, wherein the sub-sequence filtering module comprises:
a threshold setting unit configured to set a dynamic filtering threshold according to the numbers of occurrences of the seeds in each reference sub-sequence, a mean value of the numbers of occurrence, and/or a maximum descending gradient of the number of occurrence; and
a filtering unit configured to filter out a reference sub-sequence that does not meet the dynamic filtering threshold.

7. A filter processing device for a sequence alignment, comprising:
a memory configured to store a computer program; and
a processor configured to execute the computer program to perform the filter processing method for a sequence alignment according to claim 1.

8. A computer-readable storage medium storing a computer program, wherein the computer program is executed by a processor to perform the filter processing method for a sequence alignment claim 1.

9. The filter processing device for a sequence alignment according to claim 7, wherein the determining a reference sub-sequence to which each seed belongs according to a feature identifier of the seed and the mapping relationship comprises:
calculating a hash value of each seed; and
determining the reference sub-sequence to which each seed belongs from a filter hash table storing the mapping relationship, with the hash value of each seed as an address.

10. The filter processing device for a sequence alignment according to claim 7, wherein the filtering out a reference sub-sequence that does not meet a preset condition based on the numbers of occurrences of the seeds in each reference sub-sequence comprises:
setting a dynamic filtering threshold according to the numbers of occurrences of the seeds in each reference sub-sequence, a mean value of the numbers of occurrences, and/or a maximum descending gradient of the numbers of occurrences; and
filtering out a reference sub-sequence that does not meet the dynamic filtering threshold.

11. The computer-readable storage medium storing a computer program according to claim 8, wherein the determining a reference sub-sequence to which each seed belongs according to a feature identifier of the seed and the mapping relationship comprises:
calculating a hash value of each seed; and
determining the reference sub-sequence to which each seed belongs from a filter hash table storing the mapping relationship, with the hash value of each seed as an address.

12. The computer-readable storage medium storing a computer program according to claim 8, wherein the filtering out a reference sub-sequence that does not meet a preset condition based on the numbers of occurrences of the seeds in each reference sub-sequence comprises:
setting a dynamic filtering threshold according to the numbers of occurrences of the seeds in each reference sub-sequence, a mean value of the numbers of occurrences, and/or a maximum descending gradient of the numbers of occurrences; and
filtering out a reference sub-sequence that does not meet the dynamic filtering threshold.
